(19) United States
(12) Patent Application Publication
(10) Pub. No.: US 2025/0064648 A1
(43) Pub. Date: Feb. 27, 2025

(54) Improved Moisture Management Garment

(71) Applicant: MODIBODI AUSTRALIA PTY LTD, New South Wales (AU)

(72) Inventors: Kristy CHONG, New South Wales (AU); Alice WARNER, New South Wales (AU)

(73) Assignee: MODIBODI AUSTRALIA PTY LTD, New South Wales (AU)

(21) Appl. No.: 18/726,512

(22) PCT Filed: Jan. 6, 2023
(86) PCT No.: PCT/AU2023/050003

§ 371 Date: Jul. 3, 2024

(30) Foreign Application Priority Data
Jan. 7, 2022 (AU) 2022900034

(51) Int. Cl. A61F 13/15

(52) U.S. Cl. CPC A61F 13/15252; A61F 2013/15284

(57) ABSTRACT

A launderable underwear garment comprising a natural fibre based synthetic elastane free outer garment fabric having sufficient elasticity for use in a moisture management underwear garment.

### TECHNICAL FIELD

This invention relates to launderable moisture management underwear that manage bodily fluids such as period blood and/or discharge, urine, sweat etc., whose components, or majority of components, are biodegradable/compostable in active soil in a time frame of 5 years or less.

### BACKGROUND

Femtech products including reusable/launderable moisture management garments particularly “period” underwear are increasingly popular. While such products are an improvement over tampons and disposable sanitary articles and/or underwear in terms of sustainability and eco friendliness, further improvements to eco friendliness are desirable. In particular, there is a need for greater sustainability by providing moisture management underwear that is at least partially, and more preferably, completely biodegradable/compostable, and more preferably still, fully biodegradable/compostable over a short time frame of, for example, 5 years or less, while still giving the performance of existing non-biodegradable solutions.

Currently, many moisture managements garments and period underwear products typically use synthetic components including synthetic elastane and polyester-polyurethane (“PUL”) in their construction. However, such materials are not biodegradable to any significant degree, for example, in a 5 year timeframe or less, and thus such garments contribute to landfill when the product reaches its end of life.

While a small number of biodegradable basic underwear products are available, there is no moisture management underwear product on the market wherein the majority if not all of the components of the garment are biodegradable/compostable within a time frame of 5 years or less in active soil. In particular, replacement of the necessary elastane, PUL, polyester or polyester: cotton thread, nylon labels, synthetic elastane or Spandex™/elastane materials typically used in such products makes design of eco friendly underwear products challenging. This is particularly true in the case of moisture management underwear which, by necessity, must utilise a particular combination of fabric and materials which operate functionally together in a particular way for optimal moisture management function.

Given the complexity of moisture management fabric assemblies and the need for various degrees of moisture management functionality, it is clear that it is no straightforward task to design a biodegradable/compostable moisture management underwear garment that has a high moisture management volume capability and continues to fit well on the body without sagging while withstanding frequent laundering at warm temperature of around 40° C. In addition, the underwear garment's main material must have suitable stretch and elasticity as well as breathability for consumer uptake and good moisture management functionality. Avoiding synthetics including synthetic elastane in close fitting underwear garments and even more so moisture management underwear is very challenging due to the tight form fit, stretch and elasticity required. This is further difficult given that, unlike nappies and similar incontinence pants, fasteners and the like which help retain in position are not acceptable for use in such underwear products. Importantly, to ensure moisture management functionality, a period or incontinence underwear garment must be particularly form fitting/body contour hugging by nature. In particular, sagging of any kind, but in particular around the crotch/gusset area, is unacceptable and detrimental to moisture management function as the functional gusset region must be in direct/close contact at all times with the body surfaces/areas where secure and reliable moisture management is required.

Published patent AU 2014218471 describes the original Modibodi Modifier® technology for a protective insert sewn or fused to crotch portion of the outer performance fabric of a standard non-biodegradable underwear garment to provide it with moisture management capability. The three layer fabric assembly is sewn or fused into the underwear garment and is hidden by the outer performance fabric of the underwear which is said to be a bamboo/cotton blend, a merino wool jersey or in some examples a merino wool fabric [see paragraph [0062]]. However, in the Modibodi commercial underwear products, an elastic/stretchy bamboo/synthetic elastane blend jersey fabric provides the required elasticity for the reliable close fit needed to avoid sagging and associated leaking. After recent testing, the bamboo/elastane blend jersey used is now known to have a stretch recovery of about 2% of the original fabric size after 30 minutes post-stretch rest. Notably AU 2014218471 do not the degree of elasticity required to avoid sagging/leaking problems and indeed this problem was not appreciated at the filing date of AU 2014218471. There is no teaching in AU 2014218471 of how to avoid such sagging particularly for an outer garment fabric comprising a fabric/natural fibres which are synthetic elastane-free. Further, AU 2014218471 uses a moisture barrier film that is formed from a polyester layer provided with a laminate of a polyurethane film (PUL) which, due to the polyester layer, is not biodegradable. While many natural fibres have crimp, not all crimped natural fibres will have the degree of elasticity needed to ensure performance substantially equivalent to synthetic bamboo/elastane blend jersey, which is now known to have a stretch recovery stretch recovery of about 2% of the original fabric size after 30 minutes post-stretch rest. There is nothing in this document that suggests that this is degree of elasticity required for a reliable fit much less which natural fabrics would give equivalent performance.

Published patent application US 2014/0257231 describes a nappy/incontinence pants that has a reusable outer cover which is used with disposable inserts. However, the entire product is not launderable, only the outer cover. This document teaches (see paragraph [0106]) that the outer layer of an undergarment may be made from any weave or knit fabric forms, including stretch knits, noting that knitted textiles may be more inherently stretchy and elastic than woven or non-woven fabric and may impact a better fit, comfort and appearance to the outer cover. It also acknowledges that spandex (elastane) fibres or other elastomeric fibres can enhance the stretchability and elasticity impact a better fit, comfort and appearance to the outer cover than those without the elastomeric fibres teaching a preference to use elastane in the fabrics. However, there is no discussion of crimp or curl in fibres. There is no teaching or suggestion around the sagging problem of non-elastane fabrics in underwear, much less avoiding the sagging problem in the case of a biodegradable product which does not use elastane. Further, as this product uses fasteners to secure the product during use, there is no reliance on the outer fabric elasticity to retain the underwear garment in a close fit needed to ensure leakage free moisture management. There is nothing this document that suggests that this is degree of elasticity of about 2% of the original fabric size after 30 minutes post-stretch rest required for a reliable fit much less which natural fabrics would give equivalent performance.

Published patent application PCT/US2020/054532 describes a launderable incontinence underwear garment adapted to accommodate an absorbent component whereby the crotch fabric is expandable and allows for accommodation of expansion of the absorbent component when filled with urine. The underwear maintains good contact between the absorbent component and the body to reliably intercept adhering urine flow while avoiding holding the absorbent component too tightly against the wearer's body which would constrain its ability to expand with absorbed urine resulting non-absorption and leakage. The document does not teach or suggest how to make a biodegradable version of the product where at least the outer garment material does not comprise synthetic elastane and yet provides for a snug fit. The material of the outer garment layer of the underwear can be a knitted and/or woven fabric of natural, semi-synthetic or synthetic fibres, thread or yarn, but the knitted material should be a stretch knit material that includes elastic strands or threads that impart, or enhance, elastic extensibility or contractibility of the material such as those available from Schoelller Textil AG. In particular, the knitted fabric can also be knitted from a blend of non-elastic and elastic fibres, threads or yarns where the elastic components are formed from or include an elastomeric material such as elastane (spandex or Lycra). At the same time, the document teaches the stretchy crotch layer 130a should not be cotton or rayon as they would retain moisture and give a wet feel and that 130a should be formed from predominantly a material with good wicking properties such as polyester or a polyamide such as nylon, which are synthetic materials and not biodegradable in active soil. Further, this document teaches that it is important that the waist elastic portion of the garment is correctly selected to work with the other components to ensure snug holding properties of the materials of the crotch portion. While breathable, liquid impermeable barrier properties are required for outer layer 130b, there is no discussion or contemplation of biodegradable versions of these films. There is no teaching or suggestion around the sagging problem of non-elastane fabrics in underwear, much less avoiding the sagging problem in the case of a biodegradable product which does not use elastane. There is nothing this document that suggests that this is degree of elasticity of about 2% of the original fabric size after 30 minutes post-stretch rest required for a reliable fit much less which natural fabrics would give equivalent performance.

### STATEMENTS OF THE INVENTION

In a first aspect, the invention provides a launderable underwear garment comprising:
(i) an outer garment fabric comprising natural fibres having crimp/curl in the fibre, where said fibres are synthetic elastane-free wherein the outer garment fabric is one which after stretching by hand to the maximum possible degree without visibly damaging the fibres in a width/weft direction recovers to within 5% or less of the fabric original size, after 30 minutes of rest post stretch, said outer garment fabric corresponding to at least a crotch portion of the underwear garment, and
(ii) a moisture management fabric assembly which is entirely free of synthetic elastane and is located in at least the crotch portion of the underwear garment for wicking bodily fluid away from a wearer's skin into an absorbent fluid trapping core fabric component of the fabric assembly,
said moisture management fabric assembly comprising the absorbent fluid trapping core fabric component sandwiched between
a fluid wicking fabric component contactable against the wearer's skin and
a leakproof barrier component for protecting the garment from wicked fluid leakage from the fluid trapping core fabric component to the outer garment fabric.

A preferred leakproof barrier component is a breathable, waterproof web or film of a thermoplastic polymer for protecting the garment from wicked fluid leakage from the fluid trapping core fabric component to the outer garment fabric.

Also described is a launderable underwear garment comprising:
(i) an outer garment fabric comprising natural fibres having crimp/curl in the fibre, where said fibres are synthetic elastane-free wherein the outer garment fabric is one which after stretching by hand to the maximum possible degree without visibly damaging the fibres in a width/weft direction recovers to within 5% or less of the fabric original size, after 30 minutes of rest post stretch, said outer garment fabric corresponding to at least a crotch portion of the underwear garment, and
(ii) a moisture management fabric assembly which is entirely free of synthetic elastane and is located in at least the crotch portion of the underwear garment for wicking bodily fluid away from a wearer's skin into an absorbent fluid trapping core fabric component of the fabric assembly, said moisture management fabric assembly comprising the absorbent fluid trapping core fabric component sandwiched between
a fluid wicking fabric component contactable against the wearer's skin and
a leakproof barrier component which comprises a breathable, waterproof web or film of a thermoplastic polymer for protecting the garment from wicked fluid leakage from the fluid trapping core fabric component to the outer garment fabric.

Desirably, the garment is entirely free of fasteners such as Velcro or pop fasteners or buttons or the like, as the outer garment fabric, which recovers to within 5% or less of the fabric original size after 30 minutes of rest post stretch, has sufficient elasticity to provide the necessary reliable close and snug fit to the body needed for good moisture management performance. No other securing means are required to ensure the overall garment has a snug fit. It was not previously appreciated that natural fibre fabrics would need such a post-stretch recovery to function as well as elastane-blend fabrics such as the synthetic bamboo/elastane blend jersey used in the Modifier® products, which is now known to have a stretch recovery of about 2% after 30 minutes of post stretch rest. As the necessary elasticity required has now been determined, fasteners etc. are not required.

It will be understood that the entire underwear garment is launderable, that is, the outer garment fabric and the moisture management fabric assembly are both launderable.

Suitably, the web or film of a thermoplastic polymer is not attached to a synthetic fabric component, such as polyester. That is, preferably PUL is not included. Suitably, the web or film of a thermoplastic polymer is a standalone web or film which is not attached to a synthetic fabric component, such a polyester.

Also described herein is a launderable underwear garment comprising:
(i) an outer garment fabric comprising synthetic elastane-free natural fibres having crimp/curl in the fibre, preferably the outer garment fabric having a rib knit or jersey construction, said outer garment fabric corresponding to at least a crotch portion of the underwear garment, and
(ii) a moisture management fabric assembly located in at least the crotch portion of the underwear garment for wicking bodily fluid away from a wearer's skin into an absorbent fluid trapping core fabric component of the fabric assembly,
said moisture management fabric assembly comprising the absorbent fluid trapping core fabric component sandwiched between
a fluid wicking fabric component contactable against the wearer's skin and
a leakproof barrier component for protecting the garment from wicked fluid leakage from the fluid trapping core fabric component to the outer garment fabric.

A preferred leakproof barrier component is a breathable, waterproof web or film of a thermoplastic polymer for protecting the garment from wicked fluid leakage from the fluid trapping core fabric component to the outer garment fabric.

Also described herein is a launderable underwear garment comprising:
(i) an outer garment fabric comprising synthetic elastane-free natural fibres having crimp/curl in the fibre, preferably the outer garment fabric having a rib knit or jersey construction, said outer garment fabric corresponding to at least a crotch portion of the underwear garment, and
(ii) a moisture management fabric assembly which is entirely free of synthetic elastane and is located in at least the crotch portion of the underwear garment for wicking bodily fluid away from a wearer's skin into an absorbent fluid trapping core fabric component of the fabric assembly,
said moisture management fabric assembly comprising the absorbent fluid trapping core fabric component sandwiched between
a fluid wicking fabric component contactable against the wearer's skin and
a leakproof barrier component which for protecting the garment from wicked fluid leakage from the fluid trapping core fabric component to the outer garment fabric.

A preferred leakproof barrier component is a breathable, waterproof web or film of a thermoplastic polymer for protecting the garment from wicked fluid leakage from the fluid trapping core fabric component to the outer garment fabric.

In another aspect there is provided a launderable underwear garment comprising:
(i) an outer garment fabric comprising natural fibres having crimp/curl in the fibre which are synthetic elastane-free, wherein the outer garment fabric is one which, after stretching to the maximum possible degree without visibly damaging the fibres in a width/weft direction, recovers to within 5% or less of the fabric original size after 30 minutes of rest post stretch, said outer garment fabric corresponding to at least a crotch portion of the underwear garment, and
(ii) a moisture management fabric assembly which is entirely free of synthetic elastane and is located in at least the crotch portion of the underwear garment for wicking bodily fluid away from a wearer's skin into an absorbent fluid trapping core fabric component of the fabric assembly,
said moisture management fabric assembly comprising the absorbent fluid trapping core fabric component sandwiched between
a fluid wicking fabric component contactable against the wearer's skin and
a leakproof barrier component which comprises a breathable, waterproof web or film of a thermoplastic polymer for protecting the garment from wicked fluid leakage from the fluid trapping core fabric component to the outer garment fabric,
wherein the absorbent fluid trapping core component of the fabric assembly has a surface facing away from the wearer's skin in use which is laminated with the breathable, waterproof web or film of thermoplastic polyurethane, or
wherein the outer garment fabric of at least the crotch portion has a body facing surface in use which is laminated with the breathable, waterproof web or film of a thermoplastic polyurethane.

The undergarment comprises a front body portion, a rear body portion and a crotch portion, all of which are formed of the outer garment fabric comprising natural fibres which are synthetic elastane-free and wherein the fibres have crimp/curl in the fibre.

By “synthetic elastane-free natural fibres”, it is meant, natural fibres which are complete free of synthetic elastane. Suitably, the outer garment fabric comprising the natural fibres is completely free of synthetic elastane.

Preferably, the fabric of the outer garment fabric which has the natural fibres which are synthetic elastane-free and with crimp/curl in the natural fibre is one which, after stretching, preferably by hand, to the maximum possible degree (without breaking or damaging the fibres) in the width/weft direction, recovers to within 10% or less of the fabric original size after 30 minutes of rest post stretch, more preferably to within 7.5% or less of the fabric original size after 30 minutes of rest post stretch, most preferably to within 5% or less of the fabric original size after 30 minutes of rest post stretch. The stretch can be accomplished by gently hand stretching until maximum stretch is observed without any visible breaking or other damage to the stretched fibres.

Preferably, one or more of the fluid wicking fabric component, and the absorbent fluid trapping core fabric comprises synthetic elastane-free natural fibres. More suitably, all fabric components and more preferably still all underwear garment components are synthetic elastic-free (that is no synthetic elastic, and no synthetic elastane is used). Suitably, the synthetic elastane-free natural fibres used are digestible by one or more of bacteria, fungi or by enzymes produced by such microorganisms, particularly soil based microorganisms, particularly those found in composting soil or clay. Ideally, all fabric components are made of natural fibre-based components.

Suitably, the synthetic elastane-free natural fibres of the outer garment fabric in particular comprise natural fibres that have crimp and/or curl which imparts a natural waviness to a textile when woven to form a fabric. Typically, natural fibres having crimp and/or curl are animal-based fibres for example from animal fur, hair or fleece though some plant-based fibres such as cotton also may have a sufficient degree of crimp or curl. However, some natural fibres have more crimp/curl than others. Particularly preferred natural fibres having relatively high amounts of crimp or curl are wool derived. Therefore, in particular, wool derived natural fibres are preferred. Examples of wool derived fibres include Merino wool, cashmere wool, mohair wool, alpaca wool, camel wool, lamb's wool, angora wool, vicuna wool, llama wool, qiviut wool. Merino wool derived fibres are particularly preferred due to their particularly crimped/curly nature as well as Merino's fine fibre diameter (typically under 20 microns) which makes it a useful material for an underwear garment which is worn close to the skin.

Preferred outer garment fabrics comprise from 5% to 100% natural fibres having crimp/curl. In some embodiments, the outer garment fabrics may comprise 5%, 10%, 15%, 20%, 25%, 30%, 35%, 40%, 45%, 50%, 55%, 60%, 65%, 70%, 75%, 80%, 85%, 90%, 95% or 100% crimped/curled natural fibres from one or more sources. Where less than 100% of crimped/curled natural fibres are present, the fabric may be in the form of a fibre blend, where one or more other synthetic elastane-free natural fibres are used, for example, lyocell, particularly bamboo lyocell.

Particularly preferred outer garment fabrics are blends of lyocell (especially bamboo lyocell or wood-based pulp lyocell) and wool (especially Merino wool). Suitable such blends include from 20% to 90% lyocell: 80% to 10% wool, particularly Merino wool.

Preferred blends include from 10% to 100% wool, particularly Merino wool. Further preferred blends include from 20% to 100% wool, 30% to 100% wool, 40% to 100% wool, 50% to 100% wool, 60% to 100% wool, 70% to 100% wool, 80% to 100% wool, 90% to 100% wool, 95% to 100% wool, particularly Merino wool. Where less than 100% wool is use in the blend, the other components are natural derived synthetic elastane free fibres other than wool, particular a rayon, such as lyocell, most particularly Tencel™ (a wood pulp derived lyocell). Preferred blends include from 20% to 80% lyocell: 80% to 20% wool, particularly Merino wool. Further preferred blends include from 25% to 75% lyocell: 75% to 25% wool, particularly Merino wool. A preferred is a blend of 30% lyocell particularly Tencel™ lyocell: 70% wool, particularly Merino wool. Most preferred is a blend of 70% lyocell particularly Tencel™ lyocell: 30% wool, particularly Merino wool.

Preferably, the absorbent fluid trapping core fabric of the fabric assembly has a surface facing away from the wearer's skin in use which is laminated with the breathable, waterproof web or film of thermoplastic polymer such as a thermoplastic polyurethane (TPU, for example, a polyester based TPU) and most preferably a biobased thermoplastic polymer such as a biobased thermoplastic polyurethane (bTPU, having one or more biobased/digestible building blocks in the polymer). Preferred TPUs or bTPUs are 100% compostable in active soil within 3 to 5 years.

Preferably, the outer garment fabric of at least the crotch portion has a body facing surface in use which is laminated with the breathable, waterproof web or film of a thermoplastic polyurethane.

Preferably, the outer garment fabric of the fabric assembly is the same fabric material as other parts of the underwear body.

Preferably, the outer garment fabric of the fabric assembly is an integral part of a main underwear garment body outer garment fabric. That is, the outer garment fabric of the fabric assembly and the remainder of the undergarment main/body fabric are the same and preferably are of a single piece.

Preferably, the fabric assembly is insertable into the underwear garment at the crotch portion thereof.

Preferably, the web or film of the thermoplastic polymer is thermoplastic polyurethane.

Preferably, the web or film of the thermoplastic polymer is plasticizer free and/or organic or volatile solvent free. Preferably, the web or film of the thermoplastic polymer is of a biodegradable and/or compostable thermoplastic polymer having microbe digestible biobased building blocks therein. Preferably, the web or film of the thermoplastic polymer is biodegradable/biobased thermoplastic polyurethane (bTPU) having microbe digestible biobased building blocks therein. Preferred bTPU is digestible by one or more of bacteria, fungi or by enzymes produced by such microorganisms.

Preferably, the absorbent fluid trapping core fabric component is of a fabric with a terry/towel construction or non-microfibre terry construction fabric, or non-microfibre jersey construction fabric, most preferably a non-microfibre terry construction fabric.

Preferably, the absorbent fluid trapping core fabric component is selected from the group of terry/towel fabrics consisting of: Merino wool, wool, cotton, organic cotton, bamboo lyocell, bamboo viscose, other cellulosic fabric, hemp, silk, lyocell, viscose rayon, and blends thereof.

Preferably, the absorbent fluid trapping core fabric component comprises is bamboo viscose terry/towel, preferably 100% bamboo viscose terry/towel or bamboo lyocell terry/towel.

Preferably, the absorbent fluid trapping core fabric component is in the form of more than one layers of the fabric.

Preferably, the absorbent fluid trapping core fabric component is in the forms of more than one layers of the fabric, each layer having the same shape and dimensions, most preferably a gusset shape of predetermined dimensions.

In a second aspect, the invention provides a launderable underwear garment comprising:
(i) a launderable outer garment fabric comprising natural fibres having crimp and/or curl in the fibre, and which fabric and fibres are synthetic-elastane free, wherein the outer garment fabric is one which after stretching by hand to the maximum possible degree without visibly damaging the fibres in a width/weft direction recovers to within 5% or less of the fabric original size after 30 minutes of rest post stretch, and said outer garment fabric corresponding to at least a crotch portion of the underwear garment, and
(ii) a launderable moisture management fabric assembly located in at least the crotch portion of the underwear garment for wicking bodily fluid away from a wearer's skin into an absorbent fluid trapping core fabric component of the fabric assembly,
said moisture management fabric assembly comprising the absorbent fluid trapping core fabric component sandwiched between
a fluid wicking fabric component contactable against the wearer's skin and
a leakproof barrier component which comprises a breathable, waterproof web or film of a thermoplastic polymer for protecting the garment from wicked fluid leakage from the fluid trapping core fabric component to the outer garment fabric.

In a related aspect, there is provided a launderable underwear garment comprising:
(i) an outer garment fabric comprising a blend of lyocell and Merino wool fibres and having a rib knit construction, said outer garment fabric corresponding to at least a crotch portion of the underwear garment, and
(ii) a moisture management fabric assembly located in at least the crotch portion of the underwear garment for wicking bodily fluid away from a wearer's skin into an absorbent fluid trapping core fabric component of the fabric assembly,
said moisture management fabric assembly comprising the absorbent fluid trapping core fabric component sandwiched between
   a fluid wicking fabric component comprising 100% Merino wool fibres contactable against the wearer's skin and
   a leakproof barrier component which comprises a breathable, waterproof web or film of a thermoplastic polymer for protecting the garment from wicked fluid leakage from the fluid trapping core fabric component to the outer garment fabric.
wherein the fluid trapping core fabric component comprises one or more layers of a bamboo viscose terry fabric.

In a third aspect, the invention provides a launderable underwear garment comprising:
(i) a launderable outer garment fabric composed of a blend of 70% lyocell and 30% Merino wool and having a 1×1 rib knit construction, said outer garment fabric corresponding to at least a crotch portion of the underwear garment, and
(ii) a launderable moisture management fabric assembly located in at least the crotch portion of the underwear garment for wicking bodily fluid away from a wearer's skin into an absorbent fluid trapping core fabric component of the fabric assembly,
said moisture management fabric assembly comprising the absorbent fluid trapping core fabric component sandwiched between
   a fluid wicking fabric component contactable against the wearer's skin and
   a leakproof barrier component which comprises a breathable, waterproof web or film of a thermoplastic polymer for protecting the garment from wicked fluid leakage from the fluid trapping core fabric component to the outer garment fabric.

In a related aspect, there is provided a launderable underwear garment comprising:
(i) an outer garment fabric composed of a blend of 70% lyocell and 30% Merino wool and having a rib knit construction, said outer garment fabric corresponding to at least a crotch portion of the underwear garment, and
(ii) a moisture management fabric assembly located in at least the crotch portion of the underwear garment for wicking bodily fluid away from a wearer's skin into an absorbent fluid trapping core fabric component of the fabric assembly,
said moisture management fabric assembly comprising the absorbent fluid trapping core fabric component sandwiched between
   a fluid wicking fabric component composed of 100% Merino wool contactable against the wearer's skin and
   a leakproof barrier component which comprises a breathable, waterproof web or film of a thermoplastic polymer for protecting the garment from wicked fluid leakage from the fluid trapping core fabric component to the outer garment fabric.
      wherein the fluid trapping core fabric component comprises one or more fabric layers, each fabric layer composed of 100% bamboo viscose terry fibres.

In other embodiments, the invention provides a launderable underwear garment comprising a main garment body outer garment fabric, a crotch portion, and a moisture management fabric assembly for wicking bodily fluid away from a wearer's skin into an absorbent fluid trapping core fabric of the assembly, the fabric assembly comprising:
the absorbent fluid trapping core fabric sandwiched between a fluid wicking fabric component contactable against the wearer's skin and a leakproof barrier component for protecting the garment from fluid leaks from the absorbent fluid trapping core fabric,
wherein
the fluid wicking fabric component comprises a wicking fabric comprising synthetic elastane-free natural fibres;
the absorbent fluid trapping core fabric comprises one or more layers of a moisture absorbent fabric comprising synthetic elastane-free natural fibres, and wherein the leakproof barrier component comprises a fabric comprising synthetic elastane-free natural fibres, said fabric having a rib knit construction,
wherein the leakproof barrier component has a body facing surface which is laminated with a breathable, waterproof web or film of a thermoplastic polyurethane,
wherein the fluid wicking fabric component, the absorbent fluid trapping core fabric, the leakproof barrier component and the outer garment fabric are sandwiched together to form the fabric assembly;
whereby the leakproof barrier component traps bodily fluid wicked in the absorbent fluid trapping core fabric preventing leaking through to the outer garment fabric, and
wherein the main garment body outer garment fabric is the same as the outer garment fabric of the fabric assembly, and wherein the outer garment fabric the of the fabric assembly corresponds to the crotch portion of the garment.

In another aspect, the invention provides a launderable underwear garment comprising a main garment body outer garment fabric, a crotch portion, and a moisture management fabric assembly for wicking bodily fluid away from a wearer's skin into a absorbent fluid trapping core fabric of the assembly, the fabric assembly comprising:
the absorbent fluid trapping core fabric sandwiched between a fluid wicking fabric component contactable against the wearer's skin and a leakproof barrier component for protecting the garment from fluid leaks from the absorbent fluid trapping core fabric,
wherein the fluid wicking fabric component comprises a wicking fabric comprising synthetic elastane-free natural fibres;
the absorbent fluid trapping core fabric comprises one or more layers of a moisture absorbent fabric comprising synthetic elastane-free natural fibres, and wherein the leakproof barrier component comprises a fabric comprising synthetic elastane-free natural fibres, said fabric having a rib knit construction,
wherein the absorbent fluid trapping core fabric has a surface facing away from the wearer's skin which is laminated with a breathable, waterproof web or film of a thermoplastic polyurethane,
wherein the fluid wicking fabric component, the absorbent fluid trapping core fabric, the leakproof barrier component and the outer garment fabric are sandwiched together to form the fabric assembly;
whereby the leakproof barrier component traps bodily fluid wicked in the absorbent fluid trapping core fabric preventing leaking through to the outer garment fabric, and
wherein the main garment body outer garment fabric is the same as the outer garment fabric of the fabric assembly, and the outer garment fabric of the fabric assembly corresponds to the crotch portion of the garment.

In another aspect, the invention provides a launderable underwear garment comprising a main garment body outer garment fabric, a crotch portion, and a moisture management fabric assembly for wicking bodily fluid away from a wearer's skin into an absorbent fluid trapping core fabric of the assembly, the fabric assembly comprising:
the absorbent fluid trapping core fabric sandwiched between a fluid wicking fabric component contactable against the wearer's skin and a leakproof barrier component for protecting the garment from fluid leaks from the absorbent fluid trapping core fabric, wherein
the fluid wicking fabric component consists of: 100% Merino wool;
the absorbent fluid trapping core fabric comprises one or more layers of 100% bamboo viscose terry fabric, and
wherein the leakproof barrier component comprises a fabric composed of a blend of 70% lyocell and 30% Merino wool having a 1×1 rib knit construction, wherein the leakproof barrier component has a body facing surface which is laminated with a breathable, waterproof web or film of a thermoplastic polyurethane, wherein
the fluid wicking fabric component, the absorbent fluid trapping core fabric and the leakproof barrier component are sandwiched together to form the assembly, the leakproof barrier component traps bodily fluid wicked in the absorbent fluid trapping core fabric preventing leaking through to the outer garment fabric, the main garment outer garment fabric which is the same fabric as the outer garment fabric of the fabric assembly, and the outer garment fabric of the fabric assembly corresponds to the crotch portion of the garment.

In another aspect, the invention provides a launderable underwear garment comprising a main garment outer garment fabric, a crotch portion, and a moisture management fabric assembly for wicking bodily fluid away from a wearer's skin into an absorbent fluid trapping core fabric of the assembly, the fabric assembly comprising:
the absorbent fluid trapping core fabric sandwiched between a fluid wicking fabric component contactable against the wearer's skin and a leakproof barrier component for protecting the garment from fluid leaks from the absorbent fluid trapping core fabric, wherein
the fluid wicking fabric component consists of: 100% Merino wool;
the absorbent fluid trapping core fabric comprises one or more layers of 100% bamboo viscose terry fabric, and
wherein the leakproof barrier component comprises a fabric composed of a blend of 70% lyocell and 30% Merino wool having a 1×1 rib knit construction,
wherein the absorbent fluid trapping core fabric has a surface facing away from the body which is laminated with a breathable, waterproof web or film of a thermoplastic polyurethane, wherein
the fluid wicking fabric component, the absorbent fluid trapping core fabric and the leakproof barrier component are sandwiched together to form the assembly, the leakproof barrier component traps bodily fluid wicked in the absorbent fluid trapping core fabric preventing leaking through to the outer garment fabric, the main garment outer garment fabric which is the same fabric as the outer garment fabric of the fabric assembly, and the outer garment fabric of the fabric assembly corresponds to the crotch portion of the garment.

Preferably, the thermoplastic polyurethane is a biodegradable thermoplastic polymer or a biobased thermoplastic polyurethane (bTPU) comprising one or more microbe digestible or enzymatically digestible biobased/derived building blocks. (See, for example, Polymers 2019, 11, 1121; doi: 10.3390/polym11071121).

Preferably, the rib knit construction is a 1×1 rib knit construction, a 2×1 rib knit construction, a 2×2 rib construction, or a 1×2 rib construction. A 1×1 rib knit construction is preferred for underwear garments as this is the finest construction, whereas the others are less preferred as they may be too heavy for underwear garments.

Desirably, at least 95% or greater of the underwear garment is biodegradable and/or compostable in active soil in 5 years or less. Desirably, at least 97% or greater of the underwear garment is biodegradable and/or compostable in active soil in 2 years or less. Desirably, at least 99% or greater of the underwear garment is biodegradable and/or compostable in active soil in 2 years or less. Desirably, at least 100% of the underwear garment is biodegradable and/or compostable in active soil in 2 years or less.

### BRIEF DESCRIPTION OF THE DRAWINGS

Further features of the present invention are more fully described in the following description of several non-limiting embodiments included solely for the purposes of exemplifying the present invention. The following description is not a restriction on the broad summary, disclosure or description of the invention as set out above and is made with reference to the accompanying drawings in which:

FIG. 1 shows the degradation trend for bamboo viscose elastane fabric in terms of breaking load reduction with time;

FIG. 2 shows the degradation trend for the TPU film in terms of breaking load reduction with time;

FIG. 3 shows the degradation trend for the Tencel-wool fabric in terms of breaking load reduction with time;

FIG. 4 shows the visual degradation of the black fabric (95% bamboo viscose: 5% elastane) at the end of the 4th week. At the end of week 4, the black fabric sample showed over 90% of degradation where the cellulose part of the bamboo viscose component of the fabric significantly disintegrated showing over 90% degradation, and synthetic (elastane) part of the fabric structure remains intact demonstrated by clear exposure of the synthetic elastane filaments in a web structure, as can be seen in FIG. 4; and

FIG. 5 shows the strength loss (%) of all of the samples tested whereby a lower strength loss indicates a lower degree of degradation. FIG. 5 shows that bamboo viscose elastane fabric lost over 90% of its initial strength. The remaining strength arises from the synthetic elastane part of the fabric as the cellulosic part was disintegrated completely. The Tencel-Wool fabric samples did not show significant disintegration. However, it lost almost 40% of its initial strength after 4 weeks of soil burial. On the other hand, the film sample did not show a clear sign of degradation with minor loss of strength. This minor strength change suggests that it will take long time for the film biodegradation; and

FIG. 6 shows degradation of a preferred moisture management underwear garment of the invention after burial in active soil for 8 weeks. A very significant degree of damage/degradation to the main garment fabric, in particular, is evident.

### DETAILED DESCRIPTION OF THE INVENTION

The invention relates to a launderable (reusable) garment, particularly underwear, having moisture management functionality. The underwear garment of the invention includes a majority of components which are synthetic elastane free. Preferred underwear garments of the invention include components that are all synthetic elastane free. A preferred underwear garment of the invention has a fabric insert/moisture management fabric assembly and a main garment material that is synthetic elastane free.

By using synthetic elastane free naturally derived fibre-based fabrics as described herein, the preferred moisture management underwear garments of the invention are biodegradable in the sense that they are at least 80% compostable but more preferably at least 95% to 100% compostable in active soil in 5 years or less, preferably in 4 years or less, preferably in 3 years or less, more preferably in 2 years or less, and most preferably in 1 year or less. Particularly preferred garments of the invention are at least 97% compostable but more preferably at least 95% to 100% compostable in active soil in 5 years or less, more preferably 2 years or less. Most preferred garments of the invention are 100% compostable in active soil in 5 years or less, more preferably 2 years or less. Active soil comprises typical soil microbes including bacteria and fungi.

Compostable materials readily break down quickly and safely in a composting system through the action of microbial activity in a defined time period to form compost or humus comprising natural materials only which cause no harm to the environment. In contrast, moisture management underwear having elastane in the fabrics of the garment are not compostable in active soil within that timeframe. Likewise, underwear garments using polyester based materials are not biodegradable/compostable in active soil within these timeframes of interest.

In designing such a moisture management underwear garment, it was a challenge to find suitable replacement materials that preserve the moisture management functionality required and that do not sag, yet while avoiding elastane or other synthetic elastic fibres. Not only must synthetic elastane free fabrics be able to withstand frequent laundering at warm temperature of around 40° C., but it is also important that the garment main body material has sufficient suitable stretch for use in underwear, and ideally, breathability for consumer uptake. In short, avoiding synthetic elastane material in close fitting moisture management underwear garments is very challenging due to the form fit, stretch and elasticity required in an underwear garment, particularly a moisture management underwear garment. The problem is that for optimal functionality, moisture management underwear must be form fitting/body contour hugging to ensure a close fit to the body, good function, and avoidance of leakages before the moisture is wicked from the skin into the fabric assembly. Sagging of any kind in any area of underwear is undesirable, but, sagging around the crotch/gusset area of a moisture management underwear garment is particularly unacceptable as sagging is detrimental to the moisture management function. Sagging means the functional moisture management gusset region is not in the necessary direct contact with the body surfaces/areas where moisture management functionality is required, and consequently could permit moisture leakage. Furthermore, finding a biodegradable moisture barrier that retains moisture trapped in the core was a further non-trivial problem to be solved.

Lack of Elasticity in Fibres and Fabrics without Synthetic Elastane

The commercial Modibodi Modifier® products use an outer garment fabric that is a 95% bamboo/5% elastane blend which recovers after stretching tests to within 2% of its original size. Any biodegradable fabric would have to a similar elasticity for consumer acceptance in terms of suitable/equivalent performance to Modifier®. The nature of the sagging problem for natural-only fabrics was not appreciated until a biodegradable product was contemplated and tested. There was no suggestion of a solution to the problem until the minimal degree of elasticity required was determined during efforts to address this problem for the biodegradable product.

While many natural fibres have crimp and some degree of known elasticity, not all crimped natural fibres will have the degree of elasticity needed to ensure performance substantially equivalent to synthetic bamboo/elastane blend jersey of Modifier® which is now known to have a stretch recovery of about 2% after 30 minutes of post-stretch rest. There is nothing in the prior art that suggests which natural fabrics and which fibre constructions would give substantially equivalent performance (defined as 5% or less recovery after 30 minutes of post-stretch rest) to the synthetic elastic outer garment fabric blend used in the commercially available Modifier® products. While the Applicant's AU 2014218471 teaches that the Merino wool of the first (inner layer) possesses a natural crimp which allows air to flow through to the intermediate layer to assist in drying (see paragraph [0058]), there is no discussion at all regarding elasticity much less the elasticity required to provide an equivalent performance to the elastic outer garment fabric blend used in the Modifier® products.

The inventors tested several types of fabrics constructed of naturally derived fibres (e.g. 100% organic cotton in various ribs and jersey, Merino jersey, a bamboo/cotton blend) that do not contain synthetic elastane but which were initially expected to have sufficient inherent stretch and elasticity to function well in a synthetic elastane free moisture management underwear garment. However, these fabrics when tested and used to made moisture management underwear resulted in a garment that sagged when worn for longer than about 30 minutes, particularly at the gusset/crotch region of the underwear which, as explain above, is it the critical part where sagging cannot be tolerated. Surprisingly and most unexpectedly, the inventors found that fabrics of natural fibres or blends of natural fibres with crimp which have a stretch recovery of about 5% after 30 minutes of post stretch rest would give an equivalent performance to the synthetic elastic outer garment fabric used in the commercially available Modifier® products. For example, in some embodiments, this performance was achieved using a knit fabric of rib knit construction, particularly 1×1 rib and most particularly 1×1 rib comprising natural fibres having crimp/curl as the main garment outer/body fabric material (preferably corresponding to the outer layer of the moisture management fabric insert) resulted in a moisture management underwear garment that had the least sagging throughout the underwear garment, but in particular sufficiently minimal sagging at the gusset area which is key for functionality.

While knit fabrics having a rib knit construction are known to have some stretch in one direction (the width/weft direction) and some degree of recovery, they are also known to tend to lose stretch and elasticity/recovery relatively quickly after being worn for a short period of time (e.g., an hour or two). For this reason, the inventors initially considered that use of a rib knit construction fabric would have been the least successful in terms of providing an underwear garment that had sufficiently little sagging to maintain moisture management function through a 12 or 24 hour period of wearing. Indeed, for quality control purposes, any main underwear garment fabric has to have a recovery after maximum stretch in the width/weft direction of within 10% or less, more preferably 5% or less of the originally size/dimensions prior to stretching. This is very challenging given the need for a comfortable and not too thick material required for an underwear garment which is worn next to the skin. It was not at all expected that a moisture management material could be made using a rib knit construction fabric having synthetic elastane free fibres which have crimp and/or curl as the main fabric of the underwear garment. In particular, the inventors were surprised to discover that a 100% wool fabric such as Merino having a jersey construction would recover to within 7% or less of the original dimensions after maximum weft stretching. The inventors were further surprised to discover that a 1×1 rib construction fabric having some wool fibre content would recover even better to within 5% or less of the original dimensions after maximum weft stretching. It was completely unexpected that a 1×1 ribbed fabric being a blend of Merino would have similar stretch and recover as the 95% bamboo viscose: 5% elastane jersey fabric as the outer garment fabric used in the Modifier underwear product. Furthermore, unexpectedly, the biodegradable/compostable fabric assembly described herein was found to have better air permeability than the corresponding/equivalent fabric assembly made from elastane-containing fabrics.

Therefore, the invention relates to a preferred underwear garment of the invention comprising a main garment outer garment fabric, a crotch portion, and a moisture management fabric assembly for wicking bodily fluid away from a wearer's skin.

Fluid wicked away from the wearer's skin is wicked into an absorbent fluid trapping core fabric of the assembly.

Suitably, the fabric assembly comprises a structure where the absorbent fluid trapping core fabric is sandwiched between a fluid wicking fabric component contactable against the wearer's skin and a leakproof barrier component for protecting the garment from fluid leaks from the absorbent fluid trapping core fabric.

Preferably, the fluid wicking fabric component comprises a wicking fabric comprising synthetic elastane-free natural fibres.

Preferably, the absorbent fluid trapping core fabric comprises one or more layers of a moisture absorbent fabric comprising synthetic elastane-free natural fibres.

Preferably, the leakproof barrier component comprises a fabric comprising synthetic elastane-free natural fibres having a rib knit construction, particularly a rib, such as a 1×1 rib and most particularly 1×1 rib comprising a natural fibre having crimp/curl. In some embodiments, 2×1, 1×2 or 2×2 rib is not used as these fabrics may be too heavy for use in underwear.

In preferred embodiments, all three of the fluid wicking fabric component, the absorbent fluid trapping core fabric and the leakproof barrier component each comprise a fabric having natural fibres whereby the fabric and the fibres are free of synthetic elastane.

Preferably, the leakproof barrier component has a body facing surface which is laminated with a web or film of a thermoplastic polymer such as a thermoplastic polyurethane. This is a different material to polyester-polyurethane composite (PUL) which is not biodegradable in active soil in 5 years or less. In another embodiment, the absorbent fluid trapping core fabric may be provided with the web or film of a thermoplastic polymer such as a thermoplastic polyurethane, wherein the thermoplastic polymer is bonded to the underside of the absorbent fluid trapping core fabric, that is, the side of the absorbent fluid trapping core fabric that faces away from the user's body when the garment is worn. Advantageously, the latter arrangement reduces crinkling sensations experienced by the wear compared to the former arrangement. Preferably, the web or film is waterproof. Suitably, the web or film is breathable.

Desirably, the thermoplastic web or film is of thermoplastic polymer such as a thermoplastic polyurethane (TPU), preferably plasticizer free and more preferably biodegradable and/or compostable polymer or TPU. Such biodegradable and/or compostable TPUs are known as bioTPUs or bTPUs, and comprise biobased, microbial digestible linking building blocks in the polymer chains, for example, which may be based on polyols or polycaprolactone.

In one preferred embodiment, suitable TPU webs or films are available commercially from Ding Zing Advanced Materials Inc, for example, a TPU film available under product code FS2090.

Furthermore, thermoplastic polymers including such thermoplastic polyurethanes are preferred as they can be activated by heat and does not need toxic chemicals to be laminated or bonded to another fabric.

It will be understood that the fluid wicking fabric component, the absorbent fluid trapping core fabric, the leakproof barrier component and the outer garment fabric are sandwiched together to form the fabric assembly. The fabric assembly can be built into the garment itself or can be assembled separately and then inserted into the underwear garment. However, the former arrangement is particularly preferred.

During use, the leakproof barrier component traps bodily fluid wicked in the absorbent fluid trapping core fabric, thereby preventing leaking through to the outer garment fabric of the garment or underwear.

Desirably, the main garment outer garment fabric of the garment/underwear is the same fabric as the outer garment fabric of the fabric assembly. Preferably, the outer garment fabric the of the fabric assembly corresponds to the crotch portion of the garment/underwear.

In a preferred embodiment, the moisture absorbent fabric of the absorbent fluid trapping core fabric is fabric with a terry/towel construction, particularly a non-microfibre terry construction fabric. Alternatively, the moisture absorbent fabric of the absorbent fluid trapping core fabric may be of a non-microfibre jersey construction fabric, most preferably a non-microfibre terry construction fabric. In preferred embodiments, the moisture absorbent fabric of the absorbent fluid trapping core fabric is selected from the group of terry/towel fabrics consisting of: Merino wool, wool, cotton, organic cotton, bamboo lyocell, bamboo viscose, other cellulosic fabric, hemp, silk, lyocell, viscose rayon, and blends thereof.

Preferably, the moisture absorbent fabric of the absorbent fluid trapping core fabric is bamboo viscose terry/towel, preferably 100% bamboo viscose terry/towel. Desirably, the moisture absorbent fabric of the absorbent fluid trapping core fabric is two layers of the same fabric, preferably each layer having the same shape and dimensions, most preferably a gusset shape of predetermined dimensions.

Suitably, each layer of the moisture absorbent fabric has a density of from about 300 to about 500 gsm, preferably about 300 gsm to about 350 gsm, more preferably 320 to 330 gsm.

In a particularly preferred embodiment, the fabric of the absorbent fluid trapping core fabric comprises at least two layers of bamboo viscose terry, each layer having a density of 320 gsm.

Suitably, the rib knit construction of the fabric of the outer garment fabric has a 1×1 rib knit construction and most particularly 1×1 rib comprising a natural fibre having crimp/curl. Most preferably, the rib knit construction of the fabric of the outer garment fabric has a 1×1 rib knit construction.

Desirably, the outer garment fabric is selected from the group consisting of: Merino wool, cotton, hemp, silk, lyocell (Tencel™), bamboo lyocell, bamboo viscose, viscose rayon, and blends thereof.

In one embodiment, the outer garment fabric is selected a blend of bamboo and Merino wool. Desirably, the outer garment fabric is a blend of 70% lyocell and 30% Merino wool. Other bamboo and Merino blends that may be used as described above.

Preferably, the outer garment fabric has a density of from about 150 to about 300 gsm, more preferably 190 gsm.

Suitably, the fabric of the outer garment fabric consists of a blend lyocell and Merino wool having a density of 190 gsm.

Preferably, the fabric of the fluid wicking fabric component is selected from the group consisting of: Merino wool, cotton, bamboo lyocell, bamboo viscose, cotton, cellulosic fabric, hemp, silk, lyocell, viscose rayon, and blends thereof.

Desirably, the fluid wicking fabric component has a density of from about 180 to about 300 gsm. Preferably, the fabric of the fluid wicking fabric component has a density of 190 gsm.

Preferably, the fluid wicking fabric component consists of 100% Merino wool.

Most preferably, the fabric of the fluid wicking fabric component is Merino wool having a density of 190 gsm.

In preferred embodiments, at least one or more of the components of the fabric assembly are biodegradable and/or compostable in active soil. In preferred embodiments, the outer garment fabric of the garment is biodegradable and/or compostable in active soil within 5 years or less.

Suitably, in preferred embodiments, at least 80% or greater of the underwear garment is biodegradable and/or compostable in active soil in five years or less. Suitably, in preferred embodiments, at least 90% or greater of the underwear garment is biodegradable and/or compostable in active soil in five years or less. Suitably, in preferred embodiments, at least 95% or greater of the underwear garment is biodegradable and/or compostable in active soil in five years or less. Suitably, in preferred embodiments, at least 97% or greater of the underwear garment is biodegradable and/or compostable in active soil in five years or less. Suitably, in preferred embodiments, 100% of the underwear garment is biodegradable and/or compostable in active soil in five years or less.

Suitably, in preferred embodiments, at least 80% or greater of the underwear garment is biodegradable and/or compostable in active soil in four years or less. Suitably, in preferred embodiments, at least 90% or greater of the underwear garment is biodegradable and/or compostable in active soil in four years or less. Suitably, in preferred embodiments, at least 95% or greater of the underwear garment is biodegradable and/or compostable in active soil in four years or less. Suitably, in preferred embodiments, 100% of the underwear garment is biodegradable and/or compostable in active soil in four years or less.

Suitably, in preferred embodiments, at least 80% or greater of the underwear garment is biodegradable and/or compostable in active soil in three years or less. Suitably, in preferred embodiments, at least 90% or greater of the underwear garment is biodegradable and/or compostable in active soil in three years or less. Suitably, in preferred embodiments, at least 95% or greater of the underwear garment is biodegradable and/or compostable in active soil in three years or less. Suitably, in preferred embodiments, 100% of the underwear garment is biodegradable and/or compostable in active soil in three years or less.

Suitably, in preferred embodiments, at least 80% or greater of the underwear garment is biodegradable and/or compostable in active soil in two years or less. Suitably, in preferred embodiments, at least 90% or greater of the underwear garment is biodegradable and/or compostable in active soil in two years or less. Suitably, in preferred embodiments, at least 95% or greater of the underwear garment is biodegradable and/or compostable in active soil in two years or less. Suitably, in preferred embodiments, 100% of the underwear garment is biodegradable and/or compostable in active soil in two years or less.

Suitably, in preferred embodiments, at least 80% or greater of the underwear garment is biodegradable and/or compostable in active soil in one year or less. Suitably, in preferred embodiments, at least 90% or greater of the underwear garment is biodegradable and/or compostable in active soil in one year or less. Suitably, in preferred embodiments, at least 95% or greater of the underwear garment is biodegradable and/or compostable in active soil in one year or less. Suitably, in preferred embodiments, at least 97% or greater of the underwear garment is biodegradable and/or compostable in active soil in one year or less. Suitably, in preferred embodiments, 100% of the underwear garment is biodegradable and/or compostable in active soil in one year or less. It will be understood that the % of the garment that is biodegradable/compostable can be increased by removal of any non or poorly degradable components, for example, such as a waistband and/or leg elastic containing synthetic elastane. It will be further understood that cutting the garment into smaller pieces prior to composting will speed up the rate of degradation.

It will be understood that the % of the underwear garment that is biodegradable and/or compostable relates to the ratio of starting weight of the underwear garment to the remaining weight of the garment after the specified time frames.

Desirably, the outer garment fabric the of the fabric assembly corresponds to the gusset/crotch portion of the garment. Suitable, the underwear comprises a main garment body/article garment fabric which is the same fabric as the outer garment fabric of the fabric assembly.

In a particularly preferred embodiment, the invention provides a launderable underwear garment comprising a main garment outer garment fabric, a crotch portion, and a moisture management fabric assembly for wicking bodily fluid away from a wearer's skin into an absorbent fluid trapping core fabric of the assembly, the fabric assembly comprising:

the absorbent fluid trapping core fabric sandwiched between a fluid wicking fabric component contactable against the wearer's skin and a leakproof barrier component for protecting the garment from fluid leaks from the absorbent fluid trapping core fabric, wherein
the fluid wicking fabric component consists of: 100% Merino wool;
the absorbent fluid trapping core fabric comprises one or more layers of bamboo viscose terry fabric, and wherein the outer garment fabric component comprises a fabric composed of a blend of 70% lyocell and 30% Merino wool having a 1×1 rib knit construction,
wherein the leakproof barrier component has a body facing surface which is laminated with a breathable, waterproof web or film of a thermoplastic polyurethane, or wherein the absorbent fluid trapping core fabric has a surface facing away from the body which is laminated with a breathable, waterproof web or film of a thermoplastic polyurethane, wherein
the fluid wicking fabric component, the absorbent fluid trapping core fabric and the leakproof barrier component are sandwiched together to form the assembly, the leakproof barrier component traps bodily fluid wicked in the absorbent fluid trapping core fabric preventing leaking through to the outer garment fabric, the main garment outer garment fabric which is the same fabric as the outer garment fabric of the fabric assembly, and the outer garment fabric of the fabric assembly corresponds to the crotch portion of the garment.

As explained elsewhere herein, preferably, the thermoplastic polyurethane is a biobased thermoplastic polyurethane (bTPU) which comprises biosourced components which make the TPU amenable to degradation in active soil.

In particularly preferred embodiments, the garment, and particularly underwear of the invention further comprises one or more of:
a care label comprising a 100% natural fibre, such as 100% cellulose acetate, 100% cotton, 100% hemp, 100% cellulosic fibre, preferably, the label using a biodegradable ink;
a natural fibre thread, such as 100% cotton thread, 100% silk thread or 100% lyocell thread;
biodegradable polyamide thread;
waistband elastic comprising a blend of elastane and polyamide, such as a 11.5% elastane and 88.5% polyamide, preferably a biodegradable and/or compostable elastane and/or a biodegradable and/or compostable polyamide;
leg elastic comprising a blend of elastane and polyamide, such as a 14% elastane and 86% polyamide, preferably a biodegradable and/or compostable elastane and/or a biodegradable and/or compostable polyamide;
one or more biodegradable dyes such as plant base dyes;
trims such as cotton lace trim,
fold over labels such as 100% cotton fold over label optionally comprises a logo.

In embodiments where synthetic elastane or fabrics/fibres are used, for example, in the elastic waist and/or legbands and/or polyamide thread, it is preferred that such components are made from biodegradable versions thereof. For example, Amni Soul Eco® polyamide thread/yarn which is certified to degrade within 3 to 5 years.

Also described herein is a fabric assembly in the form of a protective insert (that is a fabric assembly) attachable to a garment, the protective insert comprising:
a first, operatively inner layer comprising a moisture-wicking fibre;
a second, intermediate layer of a moisture absorbing material;
and a third, operatively outer layer of a low-breathable, substantially moisture resistant material for protecting outer clothing in the event of a bodily discharge or inadvertent fluid leakage.

The fibre used in the first, operatively inner layer is a natural fibre, derived from natural fibre as described above. The first, operatively inner layer fibre may be hydrophilic or contain a hydrophilic compound and/or treatment. Environmentally friendly hydrophilic compound and/or treatments are preferred.

The first, operatively inner layer fibre/fabric may be in the form of a proteinaceous compound or fibre, particularly where such proteinaceous compound/fibre may be a keratinous compound/fibre Examples of keratinous compound/fibre include knitted, woven, compacted, comminuted, or reconstituted keratinous compound/fibre. Preferably, the keratinous compound/fibre is in the form of a knitted fabric. The keratinous compound/fibre may be moisture-wicking or pre-treated to enhance the moisture-absorbing capabilities thereof. The keratinous compound may be a stain-resistant compound/fibre or may be treated to make it stain-resistant. The keratinous compound/fibre may have natural antimicrobial properties or may be treated with an antimicrobial composition. Preferably, the keratinous compound/fibre may be in the form of wool, particularly Merino wool derived from the fleece of Merino sheep, as described above.

The antimicrobial composition may be an antibacterial, antiviral, or antifungal compound, or a combination of such compounds. Environmentally friendly antimicrobial compounds and/or treatments are preferred. The antimicrobial composition may be in the form of silver ions.

Alternatively, or additionally, the fabric/fibre used in the first, operatively inner layer may be a cellulosic fibre/fabric. In certain embodiments, the fabric/fibre comprise fibres derived from bamboo, cotton, hemp silk, or any combination or organic variation thereof. As such, the fibre may be a fibre commercially available under the trade names Modal™ (rayon/viscose) or Tencel™ (a wood pulp derived lyocell).

The first, operatively inner layer fibre may be included in a weight in a range of 100 gsm to 300 gsm in the protective insert, preferably between 150 gsm and 250 gsm, most preferably 190 gsm.

The first, operatively inner layer may have a thickness of between 0.5 mm and 3.0 mm, preferably between 0.8 mm and 2 mm, most preferably under 1 mm.

The intermediate layer may include an absorbent, quick drying compound or material. The intermediate layer may comprise a microfibre derived from natural sources, but not a synthetic based microfibre material. Preferred intermediate layer are manufactured from a cellulosic material, such as hemp, bamboo, cotton or combination of these naturally derived fibres.

The intermediate layer may be manufactured from a keratinous compound, such as wool, preferably Merino wool. The intermediate layer may have a thickness of between 0.5 mm and 5 mm, preferably 1-2 mm.

The third, operatively outer layer preferably does not comprise a polyester material. Most preferably, the third, operatively outer layer comprises is not a PUL type fabric, for example, commercially available under the trade name PUL. Rather, the outer layer fabric is one that has a rib knit construction or a jersey construction in either with natural fibres having crimp and/or curl as described above. In contrast to the Modifier™ fabric assembly described in AU2014218471 which uses a non-biodegradable, non-compostable PU laminated polyester waterproof layer as the outer layer of the fabric insert/assembly.

The third, operatively outer layer may have a thickness of between 0.1 mm and 2 mm, preferably between 0.2 mm and 1.5 mm, most preferably no more than 1.0 mm. The total thickness of the protective insert may be between 2 mm and 20 mm, preferably having a maximum thickness of no more than 15 mm, no more than 10 mm, more than 5 mm, most preferably having a maximum thickness of no more than 3 mm.

One or more of the layers may include one or more hypoallergenic compounds. The keratinous compound may be pre-treated to enhance the hypoallergenicity thereof. Environmentally friendly hypoallergenic compounds are preferred.

The protective insert/fabric assembly may form part of a garment, particularly an undergarment, and as such may be non-removably attached to the undergarment. The protective insert/fabric assembly may be fixed or attached to the garment in, proximal to, substantially covering, or extending over, at least part of a bodily discharge zone.

As such, the protective insert may be sewn or fused to the garment. In preferred embodiments, the outer layer of the insert/assembly correspondence to the garments main/primary out layer or material of construction.

In use, the first, operatively inner layer may be proximal to the body of a wearer, while the outer zone is then distal from a wearer.

According to another aspect of the invention, there is provided a garment having included therein a protective insert of the invention. The article of clothing may be an undergarment, particularly underwear, most preferably moisture management underwear.

In another aspect, the invention provides a protective insert for or in a garment, the protective insert comprising:
a first, operatively inner layer comprising a natural, moisture-wicking fibre in the form of Merino wool or bamboo fabric in a weight range of 150 to 250 gsm, the Merino wool fabric having a thickness of between 1 mm and 2 mm;
a second, intermediate layer of a moisture absorbing material comprising a natural microfibre, or a cellulosic material such as hemp, bamboo, cotton or combination of these naturally derived fibres, or a keratinous compound, such as wool, preferably Merino wool, the microfibre fabric having a thickness of 2 mm or less and having a saturation level of at least 30 ml per 10 cm² of microfibre fabric; and
a third, operatively outer layer, comprising a launderable outer garment fabric comprising natural fibres having crimp/curl in the fibre and which fabric and fibres are synthetic elastane-free, wherein the outer garment fabric is one which after stretching by hand to the maximum possible degree without visibly damaging the fibres in a width/weft direction recovers to within 5% or less of the fabric original size, after 30 minutes of rest post stretch, optionally having a thickness of 1 mm or less, and which is moisture impenetrable but allows for air penetration, for protecting outer clothing in the event of a bodily discharge or inadvertent fluid leakage.

According to a further aspect of the invention, there is provided a protective insert attachable to a garment, the protective insert comprising:
a first, operatively inner layer comprising a moisture-wicking fibre made of Merino wool fabric;
a second, intermediate layer comprising a natural microfibre fabric, or a cellulosic material such as hemp, bamboo, cotton or combination of these naturally derived fibres, or a fabric comprising a keratinous compound, such as wool, preferably Merino wool; and a third, operatively outer layer of a low-breathable, substantially moisture resistant material for protecting outer clothing in the event of a bodily discharge or inadvertent fluid leakage.

According to a further aspect of the invention, there is provided a method of manufacturing a garment having one or more moisture-absorbing zone, the method including the steps of:
providing a protective insert of the invention; and
attaching the protective insert to an area of a garment such that the positioning of the protective insert corresponds to a moisture leakage or odour producing area of a wearer's body.

The invention further extends to a method of manufacturing an protective insert/fabric assembly, the method including the steps of: providing a first, operatively inner layer comprising a moisture-wicking natural fibre as described hereinbefore; bringing into close contact with the first, operatively inner layer a second, intermediate layer of a moisture absorbing, quick drying material as described hereinbefore; and bringing into close contact with the intermediate layer, a third, operatively outer layer of a low breathable, substantially moisture-resistant material for protecting outer clothing in the event of a bodily discharge.

### Definitions

By “rib knit construction” it is meant a rib knit fabric in which the fabric is knitted in a vertical ridged pattern called ribbing and has one dimensional stretch which makes it suitable for use in round necks and cuffs of t-shirts, however, the elasticity or recovery is limited and such fabrics are understood to over stretch when worn for a period of time, typically a couple of hours. Ribbing is classified in terms of the number of knit stitches×number of purl stitches. For example, 1×1 ribbing has a ratio of 1:1 knit and purl stitches, whereas 2×2 ribbing has 2 knit stitches followed by 2 purl stitches.

By “synthetic elastic free” it is meant a fabric that does not comprise stretchy polyurethane or elastane fibres on their own or blended into another fabric. Polyurethane or elastane fibres are not biodegradable. Such fabrics include spandex (Lycra™) or other fabric blends comprising elastane fibres.

By “natural fabrics” or “natural fibres” it is meant fabric/fibres that are made from organic materials particularly animal or plant based fibres such as silk, wool, hemp, bamboo, lyocell, and cotton in contrast to synthetic fabrics/fibres which are scientifically invented are produced entirely from chemicals typically derived from coal and petroleum and include polyester, rayon, acrylic etc.

By “launderable” it is meant machine or hand washable for at least 30 times without damage or wear that destroys functionality.

### Example of Preferred Materials/Fabrics

Biodegradable/Compostable Fibres Derived from Natural Sources Such as Animals

Merino wool is a natural animal derived keratinous fibre from the fleece of the Merino breed of sheep. Merino wool is a biodegradable/compostable material which decomposed in active soil in less than a year and depending on the soil conditions, as quickly as from about 3 to 4 months or so.

Biodegradable/Compostable Cellulosic Fibres Derived from Natural Sources Such as Wood or Plants

Cellulosic fibres are derived from natural sources, and include cotton, linen, flax, hemp, jute and ramie. Man-made cellulosic fibre is called rayon which is a fibre regenerated from dissolved forms of cellulose. Cellulosic fibres which are made from natural sources of regenerated cellulose include rayon, viscose, modal, micro-modal and lyocell fibres. None of these fibres are petroleum derived fibre. They are biodegradable and typically soil compostable. Degradation in active soil is thought to be complete in 2 years or less. In the case of bamboo viscose fibres tested herein, significant degradation in active soil was found to occur after just four weeks (see below).

Rayon type fibres typically originate from wood or plant cellulose as opposed to fibres which are petroleum derived fibres.

There are three main types of rayon all generated from a natural source of regenerated cellulosic fibres mostly from made from wood pulp but also from plants such as bamboo (bamboo viscose or bamboo lyocell). Firstly, viscose rayon which made from wood pulp, modal made from beech tree pulp, and lyocell also made from beech tree pulp or eucalyptus tree pulp but using a different production process.

The difference between rayon and viscose is that rayon fabric is made via a cellulose immersion process made from wood pulp and has a high absorption capacity, while viscose is made through a cellulose xanthate process from plant fibres and has less absorption capacity.

Tencel™ is brand name for a lyocell fibre. Modal™ is a brand name for rayon/viscose from Lenzing AG that is stretched as it is made to molecules along the fibres.

By “biodegradable”, it is meant materials which can be completely compostable by (bio-assimilated) by micro-organisms such as bacteria, fungi and algae in active soil over a predetermined time frame, for example, 5 years or less, but ideally 1 year or less, when buried in active soil.

By “compostable” it is meant a material that undergoes degradation by biological processes during composting to yield CO₂, water, inorganic compounds and biomass at a rate consistent with other compostable materials and leaves no visible, distinguishable or toxic residue. The associated biological processes during composting will yield CO₂, water, inorganic compounds and biomass which leaves no visible contaminants or toxic residue/substances.

By “TPU”, it is meant a class of polyurethane plastics or more appropriately thermoplastic elastomers which exhibit elastic behaviour. In some embodiments, the thermoplastic elastomers comprise linear segmented block copolymers composed of alternating hard and soft segments. TPU typically comprises three basic components, namely, a diisocyanate, a polyol, and a chain extender. Terminal hydroxyl groups present allows for alternating blocks or segments to be inserted into the polyurethane chain. Soft segments are derived from polyols such as polyester, while the hard segments are formed from the combination of diisocyanate and a chain extender component. The chain extender is usually a small molecule with either hydroxyl or amine groups. The hard segments contribute to hardness, tensile strength, impact resistance, stiffness, and modulus while the soft segments contribute to water absorption, elongation, elasticity, softness, and degradability. Various polyurethanes with varying properties can be made by varying structures of soft and hard segments. This is why some polyurethanes are non-degradable and other undergo fast degradation. Both non-degradable and degradable polyurethanes can be designed through a proper selection of building segments. Non-degradable polyurethanes are characterized by their excellent chemical stability, abrasion resistance, and mechanical properties. Now despite the xenobiotic origins of polyurethanes, they have been found to be susceptible to degradation by naturally occurring microorganisms. In particular, degradable polyurethanes are formed by incorporating labile and hydrolysable moieties into the polymer chain. Thus, the biodegradation of polyurethanes depends on their structure, which is conditioned by several factors such as the nature of the used polyol. In particular polyester urethanes are prone to microbial degradation due to the presence of ester bonds that are known to be enzymatic hydrolysable. Under oxygen conditions, aerobic microorganisms are mostly responsible for the degradation of polymer. Biomass, carbon dioxide, and water are the final products of deterioration. As opposite to this, under anoxic conditions, anaerobic microorganisms play the main role in polymer destruction. The primary products are methane, water, and biomass.

By “bTPU”, it is meant, biodegradable TPU which is a recyclable, compostable thermoplastic polyurethane with organic blocks derived from various biobased raw materials added to the molecule chain allowing it to biodegrade in soil in between 3-5 years. Using biobased raw materials for the TPU building blocks gives bTPU properties that allow microorganisms including bacteria and/or fungi or enzymes from such microorganisms, to break it down under the right conditions. Preferably, biobased precursors obtained from biobased raw materials include one or more of biobased polyols (e.g., polycaprolactone, castor oil, soyol), biobased isocyanates (e.g., MDI, polymethylene polyisocyantes, HMDI, HDI), and biobased chain extenders (e.g., wood fibre, putrschibe diamines, piperazine diamines), for example, derived from renewable sources, including biomass, plant oils and natural fats.

In particular polyester based TPU (often derived from adipic acid esters) have poor hydrolysis resistance as well as poor microbial resistance. In contrast polyether based TPUs have strong hydrolysis and microbial resistances and are not as desirable for use in more eco-friendly moisture management underwear. Other desirable TPUs are formed from biobased starting materials to form biobased TPU from renewable resources, e.g., plant based biomass.

Desirably, the biodegradable thermoplastic polyurethane (bTPU) is formed using biobased organic blocks that are biodegradable/compostable, such that the bTPU degrades in active soil (with microbes present) in 5 years or less, preferably 4 years or less, more preferably 3 years or less, more preferably still 2 years or less, most preferably yet 1 year or less.

### Description of Preferred Embodiment

In one preferred embodiment, the main garment outer fabric is a fabric composition/blend of 70% Tencel, 30% Merino Wool with a weight of 190 gsm. The outer garment fabric has a 1×1 rib knit construction.

The top gusset fabric, that is the wicking layer is 100% Merino Wool, preferably with a Sciessent Lava XL treatment to make it odour resistance. The fabric has a weight of 190 gsm and has a single jersey construction. Wicking treatments such as MiDori wicking treatment can be added.

The middle gusset fabric (absorbent component) is 100% Bamboo Viscose Terry, preferably with a Sciessent Agion and Lava XL Slurry which is an anti-microbial and anti-odour treatment respectively. The middle gusset fabric has a weight of 320-330 gsm. The fabric has a terry toweling constructions whereby 2 layers of this fabric is used in the gusset.

The gusset waterproof/barrier layer is a biodegradable thermoplastic PU Layer from Ding Zing having a thickness of less than 1 mm. A key feature of that this layer is breathable yet waterproof.

### Example 1—Degradation on Burial in Active Soil

Four types of samples were considered-2 fabric samples, 1 TPU film and 1 underwear garment sample. The first fabric tested was a bamboo viscose elastane (black fabric) which is a blend of naturally derived cellulosic fibres from bamboo and synthetic elastane. This material is used in the main body fabric of many of the Modibodi underwear product and was chosen as a control material as it comprises a mixture of naturally derived fibres and synthetic elastane fibres. This fabric blend was chosen to demonstrate the contrast in terms of degradation in active soil between the cellulosic fibres versus the synthetic elastane. The cellulosic bamboo fibres of the fabric were expected to degrade quickly in active soil, whereas the synthetic elastane component of the fabric was not expected to degrade in soil.

The second fabric tested was a blend of 70% Tencel (lyocell) and 30% Merino wool corresponding to a blend of cellulosic fibres and animal fibres, both being naturally derived.

Biodegradability/degradation tests on these samples was tested using ISO 11721-1 method with the following modifications.
20 specimens were taken from each sample of the 2 fabric samples and the PU film sample, totaling 60 specimens. 10 specimens (10×3=30 specimens from 3 samples) were buried in active soil for 4 weeks. 10 specimens (10×3=30 specimens from 3 samples) were rested to compare strength loss.
Strength loss is measured by testing two specimens (2×3=6 specimens from 3 samples) according to ISO 13934-1 standard at the end of every week.
Observation of strength loss indicates that the same (fabric ×2 and PU) is degraded in active soil (compost from Bunnings) whereby the strength loss arises from structural degradation of the sample caused by biological agents present in the soil.
Physical degradation was studied by optical microscope to assess any degradation.
Where no significant strength loss was observed after 4 weeks, the testing was continued for an additional 4 weeks, and again at the end of eighth week for the final specimens. Where no strength loss was observed even after eighth week, the test was terminated.
Plastic boxes are used as test container. A conditioning chamber will be used for incubation, at around 100% RH.
Finally, the test was conducted on a sample of the underwear garment buried for 4 weeks and then visually analysed using an Optical Microscope after 4 weeks.
   Results of Degradation Studies after 4 Weeks Burial in Active Soil

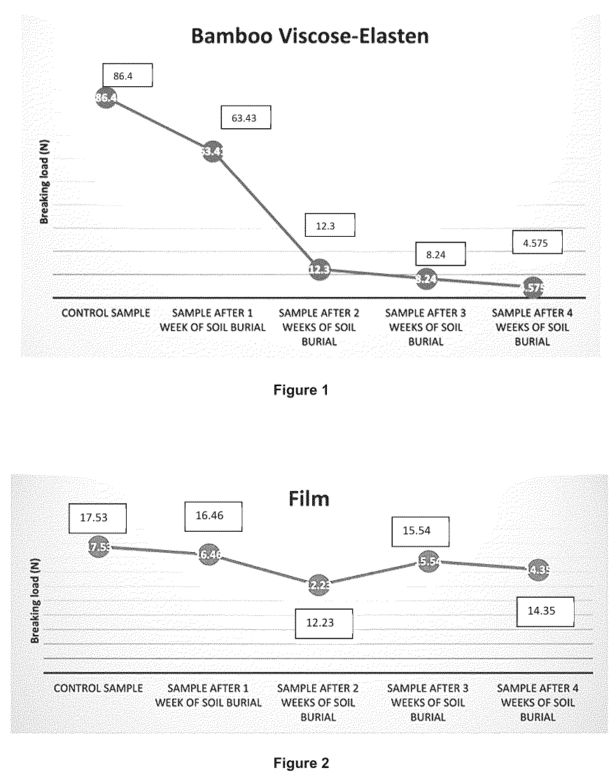

Degradation of each of the tested fabric samples (black and grey fabric samples) was observed after 4 weeks burial in active sold. The fabrics considered were (i) bamboo viscose elastane (black fabric) and (ii) Tencel Merino wool blend (pale grey fabric). As explained below, the naturally derived fibre component of each of these fabrics show clear degradation effects after just four weeks of soil burial with significant degradation of the bamboo viscose fibres of the bamboo viscose elastane fabric tested. In contrast, the PU film sample does not show a clear sign of degradation after 4 weeks of burial. The degradation trend and strength loss (%) are shown in FIGS. 1 to 3 which demonstrate a gradual loss of breaking load at the end of each week for four consecutive weeks of soil burial.

FIG. 1 shows a clear degradation trend for the bamboo viscose elastane fabric in terms of breaking load reduction with time. This contrast with the results in FIG. 2 which show a poor degradation trend for the PU film in terms of breaking load reduction with time. However, the manufacture specification indicates this particular TPU film degrades in active soil in a period of 3-5 years. Therefore, the poor results for the TPU degradation after just 4 weeks burial in active soil are not unexpected given the expected degradation time of 3 to 5 years in active soil.

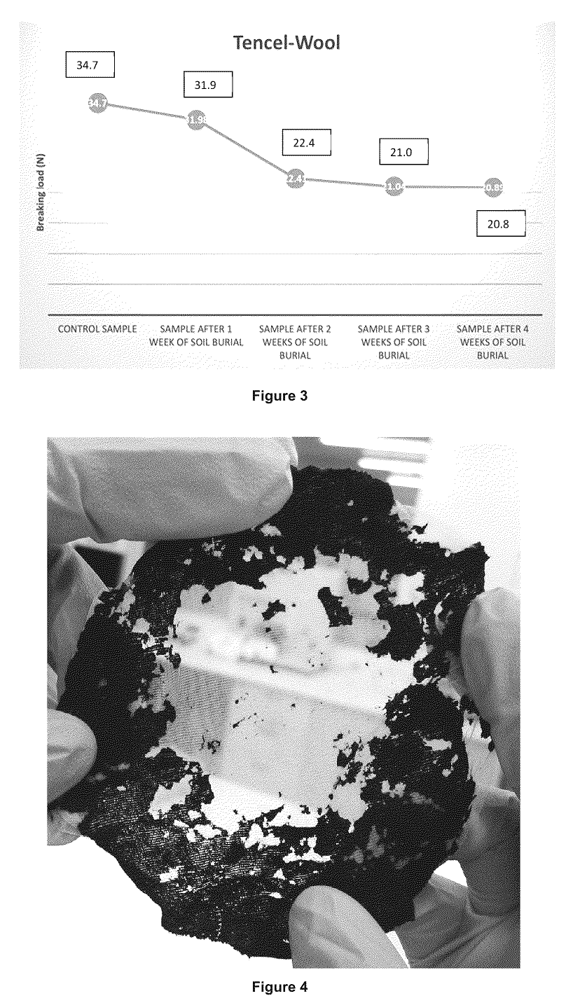

FIG. 3 shows the degradation trend for the Tencel-wool fabric in terms of breaking load reduction with time. It is evident that even after only four weeks of burial time in active soil, that the Tencel (lyocell) Merino wool blend fabric is degrading. This fabric is a preferred fabric for use in the absorbent fluid trapping core fabric of a preferred underwear garment of the invention.

FIG. 4 shows the visual degradation of the bamboo viscose elastane (black) fabric at the end of the 4th week. At the end of week 4, the black fabric sample showed over 90% of degradation where the cellulose fibres of the bamboo component had significantly disintegrated, while as expected, the synthetic elastane fibres of the fabric structure remain intact, as demonstrated by clear exposure of the synthetic elastane filaments in a web structure, as can be seen in FIG. 4.

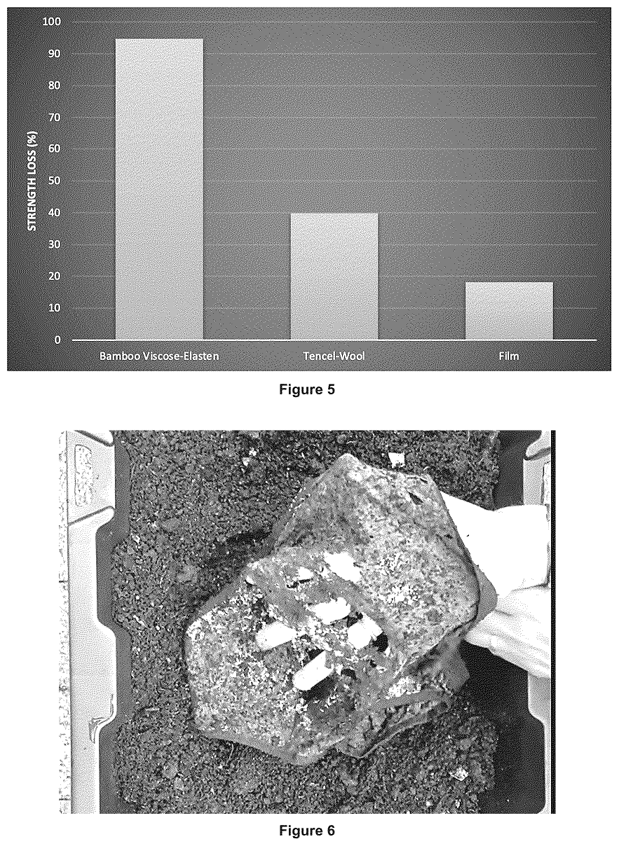

FIG. 5 shows the strength loss (%) of all of the samples tested whereby a lower strength loss indicates a lower degree of degradation. FIG. 5 shows that bamboo viscose elastane fabric lost over 90% of its initial strength. The remaining strength arises from the synthetic elastane part of the fabric as the cellulosic part was disintegrated completely. The Tencel-Wool fabric samples did not show significant disintegration. However, it lost almost 40% of its initial strength after 4 weeks of soil burial. On the other hand, the film sample did not show a clear sign of degradation with minor loss of strength. This minor strength change suggests that it will take long time for the film biodegradation.

### Experiment 2—Comparison of Absorbency Rate with Modifier/Microfibre Based Equivalent Insert/Fabric Assembly

Using a syringe filled with blood (porcine blood/saline solution), 2.5 ml of blood was added to the gusset to be tested, waiting for 5 minutes, then blood was added continuously in 2.5 ml increments, waiting 5 minutes in between each time, until saturation. Measurements of absorbency, speed of absorption, wetback, and dryness were made and recorded. All the samples were washed/dried twice and pre-conditioned for at least 24 hrs at standard atmosphere before performing the test. The pressure is required to force the water through the sample following ISO 811:2018 was noted.

### Results

The test results for absorbency tests are summarised in Table 1 which reports absorbency and backstain of the sample after adding 2 doses of liquids to the samples. The addition of the second dose of liquid was done at the same place as the first dose and absorbed quicker than the first dose for both samples.

| | Absorbency | | | Ave Pressure | |
|---|---|---|---|---|---|
| | Speed of absorbency | | | required | |
| | of 2.5 ml (seconds) | | | for leaking | |
| | 1^{st} dose | 2^{nd} dose | | (kPa) | |
| | (blood | (blood | Back- | | mm |
| | 2.5 ml) | 2.5 ml) | staining | kPa | H₂O |
| Modifier fabric | 16 ± 5 | 5 ± 2 | No B/S | 9.2 ± .4.4 | ~938 |
| assembly | | | | | |
| (Moderate Heavy, | | | | | |
| Size 14) | | | | | |
| Biodegradable Bikini | 26 ± 10 | 2 ± 1 | No B/S | 4.2 ± 0.5 | ~428 |
| fabric assembly | | | | | |
| (Moderate Heavy, | | | | | |
| Size 14) | | | | | |

After the first 2.5 ml blood addition, the Biodegradable Bikini (Moderate Heavy, Size 14) sample showed very fast absorbency (2 seconds) which was faster than the corresponding Modifier technology insert (5 seconds). Neither sample showed back staining.

### Experiment 3—Dryness Test

Dryness of the provided samples was studied after adding 2 doses of 2.5 mls as set about above. The results are reported in terms of the diameter of the wet area in tissue expression. The results are considered to depend on presence of free liquid near the surface and the absorption capacity of the underlayer. All the samples showed wet expression on tissue paper taken after 1 min of complete disappearance of liquid from the surface. Usually, the samples with poor absorption showed vivid and wider expression in tissue, indicating a wet feeling during wear. After adding a second dose of blood, the wetness increased in both cases as there were more liquid and more opportunity to spread under even small pressure.

### Results

| | Average value after | |
|---|---|---|
| | 1^{st} dose | 2^{nd} dose |
| | (blood 2.5 ml) | (blood 2.5 ml) |
| Modifier fabric assembly size 14 | 20 ± 5 | 33 ± 9 |
| Biodegradable fabric assembly size 14 | 20 ± 6 | 33 ± 6 |

Both fabric assembly compare well to each other in terms of dryness feel after moisture insult.

### Experiment 4—Air Permeability Test

Air permeability is defined as the rate of airflow passing perpendicularly through a known area under a prescribed air pressure differential between the two surfaces of a material. The airflow through an area of fabric is measured at a constant pressure drop across the fabric of 10 cm head of water. The specimen is clamped over the air inlet using rubber gaskets and air is sucked through it by a pump.

### Results

| | Average | Average | | Air |
|---|---|---|---|---|
| | flow rate | flow rate | Area | permeability |
| | (L/min) | (cm³/s) | (cm²) | (cm³/s/cm²) |
| | Airflow rate in L/min (at a pressure of 500 Pa) | | | |
| Modifier fabric | 0.4 ± 0.05 | 5.9 | 38.5 | 0.2 ± 0.01 |
| assembly size 14 | | | | |
| Biodegradable | 0.6 ± 0.02 | 9.8 | 38.5 | 0.3 ± 0.01 |
| fabric assembly | | | | |
| size 14 | | | | |

A higher value of air permeability at a certain pressure indicates better breathability of the tested fabric insert/assembly (gusset). Unexpectedly, the biodegradable/compostable gusset described herein has better air permeability than the corresponding gusset made from elastane containing fabrics.

### Experiment 5—Stretch and Recovery Testing

A 10 cm×10 cm square of each fabric considered was cut from the fabric. Fabrics used are shown in the table below.
Each fabric square was stretched to the maximum width (weft) capacity and the value of new width was recovered as the stretch measurement. Stretching may be by hand. Maximum stretch is without fibre/fabric damage/breakage being visibly observable.
The sample was allowed to recover for one minute and the recovered width was measured and recorded.
The sample was allowed to rest for 30 minutes and the recovered width was measured and recorded.

| | Fabric | | | | | | Recovery @ |
|---|---|---|---|---|---|---|---|
| | Composition | Stretch | Stretch | Recovery @ | Recovery | Recovery @ | 30 Mins |
| Knit | Starting base | (cm) | (% of | 1 Min | (% of | 30 Mins | (% of |
| Construction | width (weft) | 10 cm | original) | (cm) | original) | (cm) | original) |
| 2 × 2 Rib | 100% Organic | 29.5 | 295% | 16.5 | 165.0% | 14.8 | 148.0% |
| | Cotton | | | | | | |
| 5 × 2 Rib | 100% Organic | 29.0 | 290% | 14.3 | 143.0% | 14.0 | 140.0% |
| | Cotton | | | | | | |
| 1 × 1 Rib | 100% Organic | 25.0 | 250% | 15.0 | 150.0% | 14.0 | 140.0% |
| | Cotton | | | | | | |
| Jersey | 100% Merino | 21.0 | 210% | 11.0 | 110.0% | 10.7 | 107.0% |
| | Wool | | | | | | |
| Jersey | 100% Cotton | 16.0 | 160% | 13.0 | 130.0% | 12.3 | 123.0% |
| 1 × 1 Rib | 70% Tencel, | 31.0 | 310% | 11.0 | 110.0% | 10.5 | 105.0% |
| | 30% Merino Wool | | | | | | |
| 1 × 1 Rib | 70% Merino Wool, | 29.0 | 290% | 11.0 | 110.0% | 10.5 | 105.0% |
| | 30% Tencel | | | | | | |
| Jersey | 95% Bamboo, | 23.5 | 235% | 10.5 | 105.0% | 10.2 | 102.0% |
| | 5% Elastane | | | | | | |

Higher % stretch is favoured for allowing ease of fit on the body. Recovery after stretching should be as close to 100% as possible and higher rate of recovery is also desirable. As can be seen from the results. For the 100% cotton fabric sample, the stretchability increased from 250% (of the original size) for 1×1 rib construction to 290% and 295% for 5×2 rib and 2×2 rib respectively, however, in each case after 30 minutes, the sample had recovered to only 140%, 140% and 148% of the original size, readily demonstrating that 100% cotton rib fabric would be saggy/misshapen on the body after a short time.

The jersey construction fabric has a much-reduced stretchiness with 100% cotton jersey only stretching to 160% of the original size. The stretch was slightly better for 100% Merino jersey which went to 210% of the original size. While the recovery was better than all cases of the cotton rib fabric, the 100% Merino jersey has a far better recovery to 107% of the original size after 30 minutes. Even though the recovery is good, for quality control purposes, ideal recovery has to be within ≤5% of the original width (weft) to ensure an acceptable degree of sagging.

In contrast, the 1×1 rib blends with Merino wool performed best with a recovery of 105% of the original width (weft). Notably, the elastane fabric used in the Modifier product has a recovery of 102%.

Of the naturally based elastane free fibre fabrics, it is clear that both the 30% Merino blend or the 70% Merino blend with lyocell (Tencel) gave the best overall performance in terms of stretch and recovery with the 30% Merino having a slight edge on overall performance.

The invention claimed is:

36. A launderable underwear garment comprising:
(i) a launderable outer garment fabric comprising natural fibres having crimp/curl in the fibre and which fabric and fibres are synthetic elastane-free, wherein the outer garment fabric is one which after stretching by hand to the maximum possible degree without visibly damaging the fibres in a width/weft direction recovers to within 5% or less of the fabric original size, after 30 minutes of rest post stretch, and said outer garment fabric corresponding to at least a crotch portion of the underwear garment, and
(ii) a launderable moisture management fabric assembly which is entirely free of synthetic elastane and is located in at least the crotch portion of the underwear garment for wicking bodily fluid away from a wearer's skin into an absorbent fluid trapping core fabric component of the fabric assembly,
said moisture management fabric assembly comprising the absorbent fluid trapping core fabric component sandwiched between
a fluid wicking fabric component contactable against the wearer's skin and
a leakproof barrier component for protecting the garment from wicked fluid leakage from the fluid trapping core fabric component to the outer garment fabric.

37. The launderable underwear garment of claim 36, wherein the leakproof barrier component comprises a breathable, waterproof web or film of a thermoplastic polymer for protecting the garment from wicked fluid leakage from the fluid trapping core fabric component to the outer garment fabric.

38. The underwear garment of claim 36, wherein the outer garment fabric has a 1×1 rib knit construction or a jersey construction.

39. The underwear garment of claim 36, wherein the outer garment fabric is selected from: Merino wool, cotton, hemp, silk, lyocell (Tencel™), bamboo lyocell, bamboo viscose, viscose rayon, and blends thereof.

40. The underwear garment of claim 38, wherein the outer garment fabric comprises cotton or wool.

41. The underwear garment of claim 38, wherein the outer garment fabric is Merino wool or a blend thereof.

42. The underwear garment of claim 36, wherein the garment in its entirely is free of synthetic elastane.

43. The underwear garment of claim 37, wherein the absorbent fluid trapping core component of the fabric assembly has a surface facing away from the wearer's skin in use which is laminated with the breathable, waterproof web or film of thermoplastic polyurethane.

44. The underwear garment of claim 37, wherein the outer garment fabric of at least the crotch portion has a body facing surface in use which is laminated with the breathable, waterproof web or film of a thermoplastic polyurethane.

45. The underwear garment of claim 36, wherein the outer garment fabric of the fabric assembly is an integral part of a main underwear garment body outer fabric or.
wherein the fabric assembly is insertable into the underwear garment at the crotch portion thereof.

46. The underwear garment of claim 36, wherein the leakproof barrier component is a web or film of thermoplastic polymer, and the web or film of the thermoplastic polymer is a biodegradable or biobased thermoplastic polymer comprising one or more biobased building blocks which are digestible by microorganisms, particularly soil microorganism, or is degradable in the presence of enzymes produced by such microorganisms.

47. The underwear garment of claim 36, wherein the leakproof barrier component is a web or film of thermoplastic polymer, wherein the web or film of the thermoplastic polymer is plasticizer free and/or volatile organic solvent free, and/or is of a biodegradable and/or compostable thermoplastic polymer having microbe digestible biobased building blocks therein.

48. The underwear garment of claim 36, wherein the absorbent fluid trapping core fabric component is of a fabric with a terry/towel construction or non-microfibre terry construction fabric, or non-microfibre jersey construction fabric.

49. The underwear garment of claim 36, wherein the absorbent fluid trapping core fabric component is selected from the group of terry/towel fabrics consisting of: Merino wool, wool, cotton, organic cotton, bamboo lyocell, bamboo viscose, other cellulosic fabric, hemp, silk, lyocell, viscose rayon, and blends thereof.

50. The underwear garment of claim 49, wherein the absorbent fluid trapping core fabric component comprises bamboo viscose terry/towel.

51. The underwear garment of claim 50, wherein the absorbent fluid trapping core fabric component comprises 100% bamboo viscose terry/towel or bamboo lyocell terry/towel.

52. The underwear garment of claim 36, wherein the outer garment fabric is a blend of bamboo and Merino wool.

53. A launderable underwear garment comprising:
(i) a launderable outer garment fabric comprising natural fibres having crimp and/or curl in the fibre, and which fabric and fibres are synthetic-elastane free, wherein the outer garment fabric is one which after stretching by hand to the maximum possible degree without visibly damaging the fibres in a width/weft direction recovers to within 5% or less of the fabric original size, after 30 minutes of rest post stretch, and said outer garment fabric corresponding to at least a crotch portion of the underwear garment, and
(ii) a launderable moisture management fabric assembly located in at least the crotch portion of the underwear garment for wicking bodily fluid away from a wearer's skin into an absorbent fluid trapping core fabric component of the fabric assembly,
said moisture management fabric assembly comprising the absorbent fluid trapping core fabric component sandwiched between
a fluid wicking fabric component contactable against the wearer's skin and
a leakproof barrier component which comprises a breathable, waterproof web or film of a thermoplastic polymer for protecting the garment from wicked fluid leakage from the fluid trapping core fabric component to the outer garment fabric.

54. A launderable underwear garment comprising:
(i) a launderable outer garment fabric composed of a blend of 70% lyocell and 30% Merino wool and having a 1×1 rib knit construction, said outer garment fabric corresponding to at least a crotch portion of the underwear garment, and
(ii) a launderable moisture management fabric assembly located in at least the crotch portion of the underwear garment for wicking bodily fluid away from a wearer's skin into an absorbent fluid trapping core fabric component of the fabric assembly,
said moisture management fabric assembly comprising the absorbent fluid trapping core fabric component sandwiched between
a fluid wicking fabric component contactable against the wearer's skin and
a leakproof barrier component which comprises a breathable, waterproof web or film of a thermoplastic polymer for protecting the garment from wicked fluid leakage from the fluid trapping core fabric component to the outer garment fabric.

55. The underwear garment of claim 36, wherein at least 95% or greater of the underwear garment is biodegradable and/or compostable in active soil in 5 years or less.
